(19) United States
(12) Patent Application Publication
(10) Pub. No.: US 2009/0270335 A1
(43) Pub. Date: Oct. 29, 2009

(54) COLLYRIUM FOR DRY EYE

(75) Inventors: Frank J. Holly, Yantis, TX (US); Joel S. Echols, San Francisco, CA (US)

Correspondence Address: PATRICK R. SCANLON;PRETI FLAHERTY BELIVEAU & PACHIOS LLP, ONE CITY CENTER, PORTLAND, ME, 04112-9546, US

(73) Assignee: AQUEOUS PHARMA LIMITED, Lisbon (PT)

(21) Appl. No.: 12/441,749

(22) PCT Filed: Sep. 18, 2007
(86) PCT No.: PCT/US07/20184

§ 371 Date: Mar. 18, 2009

Related U.S. Application Data
(63) Continuation of application No. 11/901,453, filed on Sep. 17, 2007, now abandoned.
(60) Provisional application No. 60/845,433, filed on Sep. 18, 2006.

(51) Int. Cl. A61K 38/08

(52) U.S. Cl. 514/15

(57) ABSTRACT

A composition for treating dry eye includes eledoisin formulated in an ester form and a phospholipid formulated in a micellar form.

### CROSS REFERENCES TO RELATED APPLICATIONS

This application claims the benefit of U.S. Provisional Application No. 60/845,433, filed Sep. 18, 2006.

### BACKGROUND OF THE INVENTION

It is now recognized that the majority of dry eye patients (greater than two-thirds), suffer from the so-called evaporative dry eye. Upon close examination, all these patients possess an unstable tear film that results in keratoconjunctivitis sicca. A significant number of patients, however, suffer from diminished tear secretion and hence the interest in secretagogues. Secretagogues stimulate and/or enhance tear production.

There are two primary problems with the formulations presently available. One, they do not address the wide spread problem of tear film instability. Second, often they contribute to the tear film instability and actually worsen rather than improve the disease.

One secretagogue that has been proposed for use in the treatment of dry eye syndrome is eledoisin, a substance produced by the salivary glands of several octopuses. Experimental formulations of eledoisin eye drops used in clinical studies have had two major shortcomings that adversely affected the benefit of the drops. First, the eledoisin was dissolved in purified water so that its molecule exists in an extended configuration. This results in the formulation not being particularly stable and the pharmaceutical effect of the eledoisin being weakened. Second, the experimental preparations typically were preserved with benzalkonium chloride, which is known to destroy the superficial lipid layer of the tear film thereby drastically reducing tear film stability.

### SUMMARY OF THE INVENTION

The above-mentioned need is met by the present invention, one embodiment of which includes a composition comprising eledoisin formulated in an ester form and a phospholipid formulated in a micellar form.

### DETAILED DESCRIPTION OF THE INVENTION

The present invention generally relates to an aqueous collyrium that contains a proven secretagogue, an undecapeptide called eledoisin (Glu-Pro-Ser-Lys-Asp-Ala-Phe-Ile-Gly-Leu-Met-NH₂), a member of the tachykinin family, which upon topical application is found to increase both the tear secretion rate and the tear volume both in dry eye patients and people not suffering from dry eye. Side effects such as hyperemia of the conjunctiva have been observed initially in some patients but chemosis was not pronounced.

One embodiment of the present invention comprises a collyrium or eye drop that promises to be much superior in efficacy to the experimental formulations used in clinical studies. In this embodiment, eledoisin is added to an aqueous solution where phospholipid micelles are present which induce helical configuration in the central core of the eledoisin molecule. Not only is the therapeutic effect more pronounced in this configuration, but the preparation becomes much more stable in the presence of the phospholipid micelles.

In one possible embodiment, a collyrium is proposed which comprises an aqueous composition containing phospholipids (e.g. lecithin, dodecyl-phosphocholine, etc.) in a micellar form and eledoisin in an ester form in a concentration ranging from about 0.1 percent (w/w) to about 1.0 percent (w/w). The aqueous vehicle will also contain a synergistic mixture of polymers (partially and fully hydrolyzed polyvinyl alcohol) which improves the wettability of the ocular surface and sufficient level of an inert nonviscous polymer (polyvinyl-pyrrolidone) to provide an elevated oncotic pressure that will prevent chemosis and have a beneficial effect on the damaged epithelial surface of the cornea and conjunctiva.

For example, one possible composition comprises fully hydrolyzed polyvinyl acetate (i.e., polyvinyl alcohol), partially hydrolyzed polyvinyl acetate, polyvinyl pyrrolidone, a secretagogue such as eledoisin, and a micellar phospholipid. The composition can further comprise water, one or more electrolytes that contribute to the well-being of the corneal epithelium such as sodium chloride and potassium chloride, one or more preservatives, and one or more buffers.

In one embodiment, the concentration of the fully hydrolyzed polyvinyl acetate is from about 0.5 percent (w/w) to about 10 percent (w/w), the polyvinyl alcohol being about 96% to about 99% hydrolyzed; the concentration of the partially hydrolyzed polyvinyl acetate is from about 0.5 percent (w/w) to about 10 percent (w/w), the polyvinyl acetate being about 85% to 90% hydrolyzed; the concentration of the polyvinyl pyrrolidone is from about 1 percent (w/w) to about 4 percent (w/w); and the concentration of the phospholipids is about 0.005 percent (w/w) to 0.05 percent (w/w).

The collyrium can be preserved with a suitable preservative that is practically benign and is preferably not benzalkonium chloride. Buffers, such as disodium edeate dihydrate and boric acid for example, may be added to yield a pH value between about 5.0 and 8.0, and more preferably between 6.5 and 6.9. As an alternative to using a preservative, the present invention can utilize unit dose, non-preserved packaging.

In addition, the secretagoguese stabilized by the phospholipid can be supplied separately from the basic formulation and added to the basic formulation at the time of the first opening of the bottle or package containing the basic formulation.

While specific embodiments of the present invention have been described, it should be noted that various modifications thereto can be made without departing from the spirit and scope of the invention as defined in the appended claims.

What is claimed is:

1. A composition comprising:
eledoisin formulated in an ester form; and
a phospholipid formulated in a micellar form.

2. The composition of claim 1 wherein said phospholipid is lecithin.

3. The composition of claim 1 wherein said phospholipid is dodecyl-phosphocholine.

4. The composition of claim 1 further comprising fully hydrolyzed polyvinyl acetate.

5. The composition of claim 1 further comprising partially hydrolyzed polyvinyl acetate.

6. The composition of claim 1 further comprising polyvinyl-pyrrolidone.

7. The composition of claim 1 wherein the concentration of said eledoisin is about 0.1 percent (w/w) to about 1.0 percent (w/w).

8. The composition of claim 7 wherein the concentration of said phospholipid is about 0.005 percent (w/w) to 0.05 percent (w/w).

9. The composition of claim 8 further comprising polyvinyl alcohol that is about 96% to about 99% hydrolyzed, and polyvinyl acetate that is about 85% to 90% hydrolyzed, and polyvinyl pyrrolidone.

10. The composition of claim 9 wherein the concentration of said polyvinyl alcohol about 0.5 percent (w/w) to about 10 percent (w/w), the concentration of said polyvinyl acetate is about 0.5 percent (w/w) to about 10 percent (w/w), and the concentration of said polyvinyl pyrrolidone is about 1 percent (w/w) to about 4 percent (w/w).
